(19) United States
(12) Patent Application Publication
(10) Pub. No.: US 2016/0067652 A1
(43) Pub. Date: Mar. 10, 2016

(54) Nitrification-Enhanced Ammonia Scrubber for Animal Rearing Facilities

(71) Applicant: The United States of America as represented by the Secretary of Agriculture, Washington, DC (US)

(72) Inventor: Philip A. Moore, JR., Fayetteville, AR (US)

(21) Appl. No.: 14/482,131

(22) Filed: Sep. 10, 2014

(51) Int. Cl. B01D 53/84; C12M 1/12; C05F 17/02; B01D 53/58

(52) U.S. Cl. CPC B01D 53/84; B01D 53/58; C12M 25/14; C05F 17/02; B01D 2251/95

(57) ABSTRACT

The exhaust air scrubber system for animal containment facilities includes a two-stage scrubber configuration. Exhaust air from the animal containment facility flows first into a dust scrubber, which removes dust from the air and reduces the alkalinity of the exhaust air. The exhaust air then flows into an ammonia scrubber which removes the ammonia from the air. The ammonia is converted to nitrate by acid-tolerant nitrifying bacteria. The nitrification process produces acid, which reduces the pH of the scrubber solution in the ammonia scrubber, allowing the scrubber to capture additional ammonia.

### FIELD OF THE INVENTION

The disclosed method and apparatus relates to removing ammonia and dust from air that is exhausted from animal containment facilities. Specifically, the method and apparatus described herein relates to removing ammonia with acid produced by bacteria via nitrification.

### BACKGROUND OF THE INVENTION

The size and density of confined animal operations in the United States continues to increase, causing high levels of atmospheric emissions of compounds like ammonia gas (NH₃). Recently the U.S. EPA indicated that ammonia emissions from animal facilities will soon be regulated, since ammonia is a precursor to very small particulate matter made of ammonium sulfate or ammonium nitrate, both of which are harmful to humans.

The prior art includes several systems for treating air exhausted from animal containment facilities, including the inventor's previous U.S. Pat. No. 7,194,979 titled METHOD AND DEVICE FOR SCRUBBING AMMONIA FROM AIR EXHAUSTED FROM ANIMAL REARING FACILITIES (hereinafter “Moore ‘979), and U.S. Pat. No. 8,663,551 titled ANIMAL CONTAINMENT FACILITY VENTILATION SYSTEM (Hereinafter “Moore '551), which are hereby incorporated by reference. Both the single stage scrubber described by Moore '979 and the dual stage scrubber described by Moore '551 utilize a salt of an acidic proton donor to capture ammonia. The proton donor solution converts the volatile ammonia from the exhaust gas to non-volatile ammonium (NH₃+H⁺→NH₄⁺). The two stage system described by Moore '551 utilizes an initial scrubber to remove particulate matter or dust from the exhaust air and reduces the acid requirement for scrubbing ammonia, since much of the acid was wasted neutralizing the alkalinity associated with dust.

While the acid salts described by Moore '979 and Moore '551 are much safer than strong acids, such as hydrochloric, nitric, sulfuric and phosphoric acids, they are problematic for a variety of reasons. As ammonia is captured in the scrubber and pH increases, the iron and aluminum salts become iron and aluminum oxides and hydroxides, which can plug up pipes and cause problems within the scrubber. Sodium proton donors, such as sodium bisulfate, do not form oxides or hydroxides. However, when sodium bisulfate is used, the nitrogen-rich scrubber solution is very high in sodium, which can kill crops and cause sodic soil conditions, thereby limiting the use of this solution as a fertilizer. The best acid salt (from the fertilizer perspective) is potassium bisulfate. Its use results in the production of an ammonium potassium sulfate, which is a good fertilizer source. However, potassium bisulfate is not produced in bulk in the U.S., so it is extremely expensive.

In fact, the problem with all acids, including acid salts and strong acids, (with respect to ammonia scrubbers) is the cost. When scrubbing ammonia and dust from air exhausted from animal containment facilities, the only economic benefit to the farmer is the value of the nitrogen fertilizer captured. The inventor's research shows that the average amount of N captured (as ammonia) with a scrubber using 50 pounds of sodium bisulfate was 6.64 pounds of N. The cost of the sodium bisulfate alone rendered the process cost-prohibitive, even before consideration of the cost of the electricity, water and labor needed to operate it.

Fertilizer N, on the other hand, is fairly cheap. Strong acids are also expensive. The cheapest strong acid is generally sulfuric acid and currently it is more expensive per mole of acid than sodium bisulfate, at least when small (less than 5 gallons) quantities are purchased. Thus the need exists for a proton donor that can be used to capture ammonia exhausted from animal containment facilities which is safe for growers to use, easy to handle, safe for crops and relatively inexpensive.

The system described herein comprises a two-stage dust and ammonia scrubber wherein the particulate matter (dust) that contains alkalinity is reduced by the first scrubber, and the second scrubber reduces the amount of ammonia and volatile organic compounds (odors) from the exhaust air. The inventor's research reveals that bacteria responsible for nitrification can be used to provide an essentially unending supply of “free” acid via the nitrification process. This is an unexpected result with significant consequences.

Nitrification is a two-step process that is carried out by aerobic bacteria in soil and natural waters where ammonium (NH₄⁺) is converted to nitrate (NO₃⁻). In the first step, ammonia is oxidized to nitrite by ammonia-oxidizing bacteria (AOB), such as Proteobacteria, Nitrosomonas, and Nitrosococcus. In the second step. nitrite is oxidized to nitrate by nitrate-oxidizing bacteria (NOB), such as Nitrobacter and Nitrospira.

The reactions are as follows;

NH₄⁺+1.50₂→NO₂⁻+2H⁺+H₂O Equation 1

NO₂⁻+0.50₂→NO₃⁻ Equation 2

Thus, for every mole of ammonium oxidized, two moles of acid are formed, causing acidification. The current invention utilizes this reaction to provide the acid needed to scrub ammonia from the air exhausted from (among others) poultry or swine containment facilities.

Although most nitrifying bacteria are inhibited at low pH, the current technology utilizes a population of organisms that were slowly acclimated to acidic conditions over a period several months. This process is safe for farm personnel and can be used on any crops without worry of salt damage or other problems.

### SUMMARY OF THE INVENTION

This disclosure is directed to a scrubber system used to reduce air pollutants in the exhaust air from animal containment facilities. The system comprises a dust scrubber in series with an ammonia scrubber that uses nitrification to produce the acidity required for converting ammonia to ammonium. The bacteria responsible for nitrification are adapted to acidic conditions (pH less than 5.5).

This disclosure is also directed to a method of removing ammonia from the exhaust air from animal containment facilities. In accordance with the method, exhaust air from an animal containment facility is passed through a dust scrubber and the through an ammonia scrubber. The acidity in the ammonia scrubber is derived from nitrifying bacteria which are adapted to acidic conditions.

This disclosure is further directed to a method of making a scrubber system for an animal containment facility. In accordance with the method, an ammonia scrubber is provided and an inoculum containing ammonia-oxidizing bacteria (including, but not limited to Proteobacteria, Nitrosomonas, and Nitrosococcus) and nitrate-oxidizing bacteria (including, but not limited to Nitrobacter and Nitro spira) that have been adapted to acidic (pH<5.5) conditions is added. These bacteria, which are cultivated on a suitable substrate, (such as clay) are placed inside suitable containers which allow for the growth of biofilms within the scrubber, where they produce acid while converting ammonium to nitrate.

### BRIEF DESCRIPTION OF THE DRAWINGS

FIG. lis a schematic representation of an animal containment facility which is equipped with a scrubber system as described herein.

FIG. 2 is a more detailed schematic representation of the nitrification-enhanced ammonia scrubber described herein.

FIG. 3 is a schematic of the microcosm used for developing acid-tolerant nitrifying bacteria.

FIG. 4 is a graphical representation of the pH of the effluent water exiting the reactor used for developing acid-tolerant nitrifying bacteria.

FIG. 5 is a graphical representation of nitrate accumulation in a nitrification-enhanced ammonia scrubber.

FIG. 6 is a graphical representation of the cumulative amount of ammonium, nitrate and inorganic nitrogen captured with a nitrification-enhanced ammonia scrubber attached to a poultry house.

### DETAILED DESCRIPTION OF PREFERRED EMBODIMENTS

As schematically shown in FIG. 1, the method and apparatus described herein comprises a scrubber system 20 for an animal containment facility 22. For the purposes of this disclosure, the term “animal” includes birds (particularly poultry), mammals, reptiles, and amphibians. In the preferred embodiment, the animal containment facility 22 comprises a commercial-scale poultry containment facility. In alternative embodiments the facility 22 may house any type of animal known in the art.

The structure of the animal containment facility 22 is not critical, and the process may be practiced in any conventional facility suitable for containing animals. As generally shown in FIG. 1, air is drawn into the facility 22 through ventilating inlets 24, and exhausted through a ventilation outlet 26 in the direction of the arrow 27, and into the scrubber system 20. The scrubber system 20 comprises a water-based dust “scrubber” 28, and a nitrification-enhanced ammonia scrubber 30 arranged in series.

In the preferred embodiment, each scrubber 28, 30 is generally structured as described in Moore '551, however essentially any known scrubber may be used. For the purposes of this disclosure, a “scrubber” is defined as an apparatus which brings a liquid “scrubber solution” in contact with a gas so that impurities, contaminants, or other targeted substances are removed from the gas and retained in a resulting “product solution”.

The term “scrubber solution” is defined as the liquid used in a scrubber to remove impurities, contaminants, or other targeted substances from a gas. The term “product solution” is defined as the modified scrubber solution after the scrubber solution has been used to remove impurities, contaminants, or other targeted substances form a gas. A product solution may be recycled and reused as a scrubber solution.

In the preferred embodiment, the particulate scrubber 28 removes dust and other solids from the exhausted air. In a poultry containment facility, the dust primarily comprises dried manure, however, the particulate materials may also include microorganisms such as bacteria, fungi, and viruses, all of which can cause diseases in humans. Even the cell walls of gram negative “dead” bacteria can cause human health problems, because the dead bacteria contain large amounts of endotoxins.

The disease Histoplasmosis is caused by a fungus Histoplasma capsulatum and is promoted by “bird droppings”. The fungus is frequently found in small amounts in the lungs of human populations that are in close proximity to large poultry-containment operations. The fungus (and resulting disease) can be dangerous to poultry industry workers who are subjected to large amounts of the fungus over a prolonged period, as well as to environmentally-sensitive members of the general public who are exposed smaller amounts of the fungus. Infants, the elderly, and the immunocompromised may be particularly vulnerable. The particulate scrubber 28 described herein is designed to effectively remove essentially all of these organisms from the exhausted air.

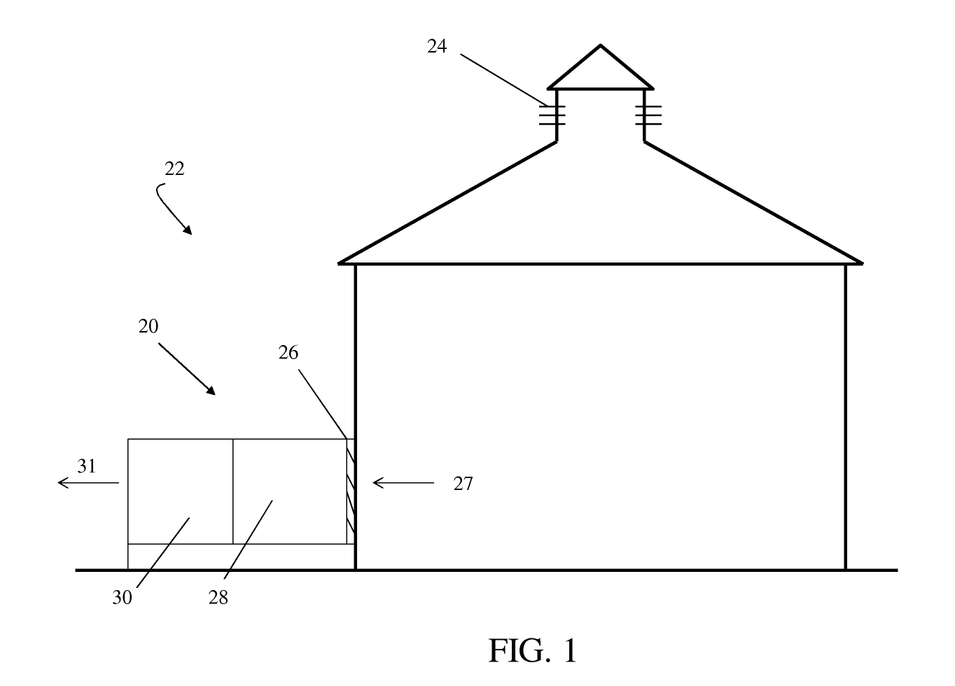

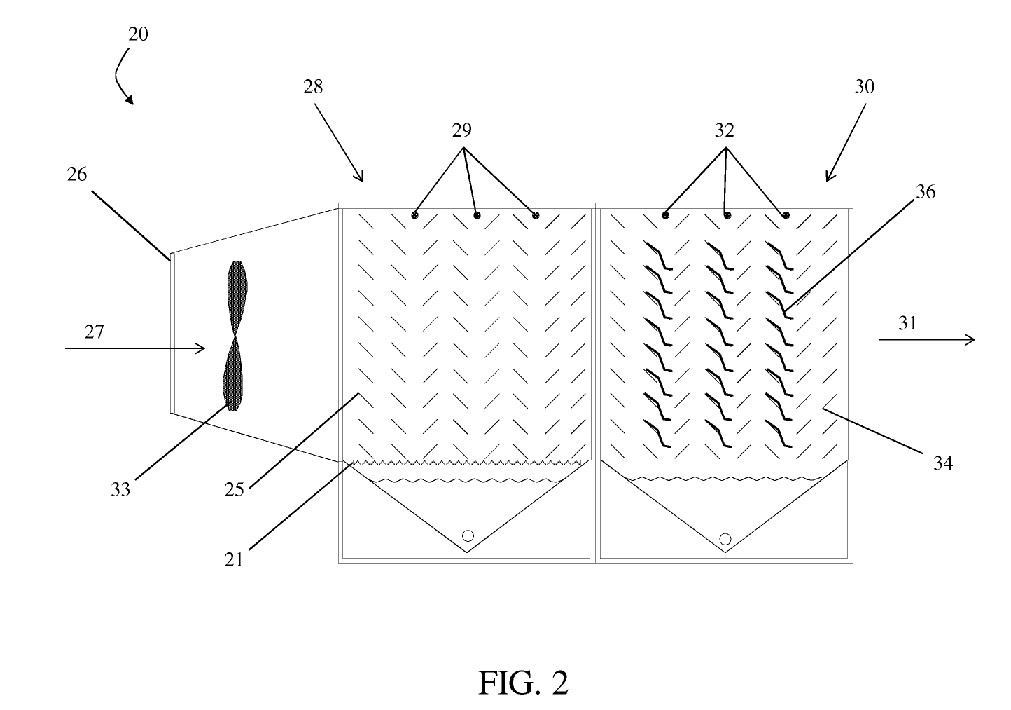

FIG. 2 shows the particulate scrubber 28 and the ammonia scrubber 30 in greater detail. With regard to the particulate scrubber, the particulate scrubber solution is essentially water that is supplied to the scrubber 28 via a plurality of nozzles 29. The water flowing through the nozzles 29 is directed to flow over a plurality of slats 25 and thereby creates a water curtain. As a fan 33 draws the exhaust air into the particulate scrubber 28, the water curtain effectively removes the manure-based dust from the exhausted air.

The removal of the dust/manure significantly reduces the alkalinity of the exhaust, as described by Moore '551. The reduction in alkalinity is critical because the ammonia scrubber 30 uses acid (protons) to convert ammonia to ammonium. Without the reduction in exhaust air alkalinity the ammonia scrubber 30 solution is prematurely neutralized and becomes ineffective at removing the ammonia from the exhausted air. The particulate scrubber 28 also includes a feather trap 21 which helps to prevent clogging of the water circulation system.

With regard to the ammonia scrubber 30, FIG. 2 shows the ammonia scrubber 30 as modified to accommodate the acid-tolerant nitrifying bacteria. The ammonia scrubber 30 utilizes acidity produced by nitrifying bacteria to capture ammonia from the exhaust air. This is a novel and distinguishing characteristic that is not present in the prior art or in the Inventor's previous patents or publications.

In the preferred embodiment, the ammonia scrubber 30 is provided with an inoculum comprising ammonia-oxidizing bacteria (including, but not limited to Proteobacteria, Nitrosomonas, and/or Nitrosococcus) and nitrate-oxidizing bacteria (including, but not limited to Nitrobacter and/or Nitrospira) that have been adapted to acidic conditions. In one embodiment, the bacteria are adapted to pH conditions below 6.5 or lower. In the preferred embodiment, the acid-tolerant bacteria are adapted to pH conditions of 5.5 or lower.

As shown in FIG. 2, similar to the dust scrubber 28, the ammonia scrubber 30 includes a plurality of nozzles 32 that direct a scrubber solution (containing acid produced by bacteria) onto a plurality of slats 34 so that an acidified water curtain is formed within the scrubber 30. Multiple specially adapted substrates 36 are attached to selected slats 34. The substrates 36 comprise a medium that accommodates and facilitates the growth of acid-tolerant nitrifying bacteria biofilm. These medium include clay or a fibrous medium (such as cheese cloth or other fabrics) or any matrix of support structures sufficient to support the biofilm. In addition to flowing over the substrates, the scrubber solution may also flow through the biofilm support structures.

In addition to ammonia, the ammonia scrubber 30 described herein also effectively removes 5-95% of the volatile organic compounds (VOCs) that are responsible for the unpleasant odors that are generally associated with commercial poultry-containment facilities.

The effectiveness of nitrifying bacteria as an acid source for the ammonia scrubber is a very surprising and unexpected result. Typically nitrification is greatly reduced when the pH is below about 6.5. However, the inventor measured very high rates of nitrification at pH values as low as 4.1 to 4.5.

Once the process is initiated, the acid-tolerant nitrifying bacteria are also present in the scrubber solution of the ammonia scrubber 30. The acid-tolerant nitrifying bacteria should be present in the scrubber solution in sufficient quantities so that the pH of the product solution is at or below 8. In the preferred embodiment, the nitrifying bacteria should be present in the scrubber solution in a sufficient concentration so that the pH level of the product solution is maintained at or below 6.5. The specific amounts/quantities of nitrifying bacteria required to meet these functional requirements will vary based on their growth rate, which will be affected by the conditions they are exposed to including temperature, dissolved oxygen, and concentrations of ammonium, nitrite and nitrate in scrubber solution.

To facilitate the growth and nitrifying activity of the bacteria, it is advantageous to maintain the scrubber and product solutions within a pre-determined temperature range. In addition to being acid-tolerant, the acid-tolerant bacteria are also cold tolerant so that the product and scrubber solutions should be maintained between 5° and 25° C. To maintain this temperature range, it may be necessary to heat the scrubber and/or product solutions. The scrubber and product solutions may be heated by any means known in the art, including solar heating or via a heat exchange process wherein heat gathered within the containment facility is used to heat the product and scrubber solutions. In one alternative embodiment, the ammonia and particulate scrubbers are positioned within the animal containment facility.

Similarly, the growth and nitrifying activity of the bacteria may be enhanced by aerating the scrubber and product solutions. The product solution may be aerated in the reservoir shown in FIG. 2 at the base of the ammonia scrubber 30, or the scrubber solution may be aerated immediately before the solution is recycled back through the nozzles 32 and onto the slats 36. The amount of aeration required (if any) depends upon the characteristics of the product and scrubber solutions, and on the operating temperature of the scrubber 30.

The scrubber system 20 shown in FIGs.1 and 2 represents a batch-type scrubber. In other words, the scrubber solution is removed periodically, (e.g. each week) and replaced with fresh water. In the preferred embodiment, the scrubber system 20 should be a “continuous flow system” whereby fresh water is slowly metered out into both the dust 28 and ammonia 30 scrubber. The product solution leaving the scrubber would flow into a pond, reservoir, irrigation canal, ditch or a man-made container and are used to irrigate/fertilize crops. This continuous flow system would keep the concentrations of metabolic products from the bacteria (nitrite, nitrate, etc.) lower than a static system, which would allow for better growth and higher nitrification rates which would produce more acidity.

As discussed above, the resulting nitrogen-rich product solution has a much lower salt content than when strong acids or acid salts are used and comprises an excellent fertilizer source. After the ammonia is removed from the exhaust, the air flows out of the ammonia scrubber 30 (in the direction of the arrow 31) and into the atmosphere or into further scrubbing/filtering or air handling devices.

In operation, as generally shown in FIGS. 1 and 2, a fan 33 draws exhaust air from an animal containment facility 22 through the ventilation outlet 26 in the direction of the arrow 27 and into a dust scrubber 28. A water-based dust scrubber 28 solution removes particulate matter (such as manure) from the exhaust air and thereby reduces the alkalinity of the exhausted air. The air then moves into an ammonia scrubber 30 that has been inoculated with acid-tolerant nitrifying bacteria that produce the acidity needed to lower the scrubber solution pH. The ammonia scrubber 30 significantly reduces the ammonia in the exhaust air. The air then flows out of the ammonia scrubber in the direction of the arrow 31. The product solutions from the dust scrubber 28 and the ammonia scrubber 30 may be recycled and re-used as scrubber solutions, or the product solutions may be directed to holding areas and subsequently used for fertilization and irrigation.

The concepts and apparatus described herein are illustrated in the following examples:

### EXAMPLE 1

### Laboratory Microcosm Study

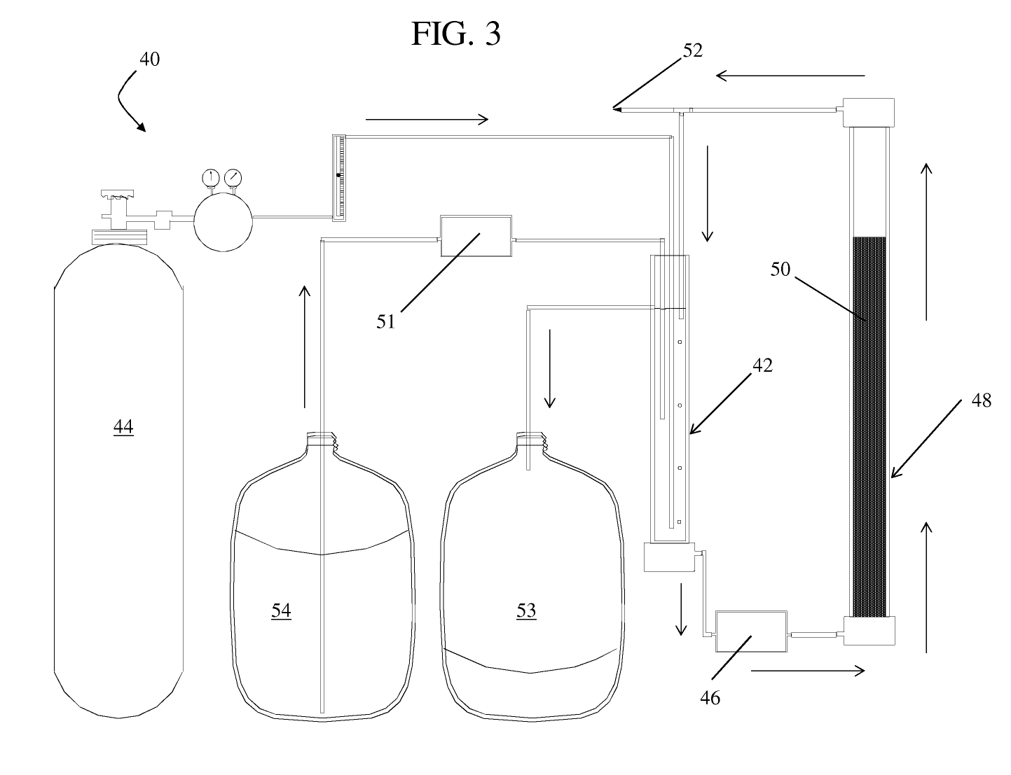

The bacteria preparation and production system 40 that was used for growing acid-tolerant nitrifiers is shown in FIG. 3. In this system, a feed solution 54 containing ammonium chloride, sodium bicarbonate and potassium phosphate was added to a mixing vessel 42 at a flow rate of approximately 7.6 ml/min. Pure oxygen was also bubbled into the mixing vessel 42 from an oxygen tank 44. This solution flowed (note that flow direction is indicated by the arrows shown in the figure) through a magnetically-driven circulation pump 46 (17 L/min capacity) into the nitrifying reactor 48 which was packed with an expanded clay substrate 50 (Filtralite HC 1.5-2.5). Both the nitrifying reactor 48 and mixing vessel 42 were made from 1.5″ ID clear acrylic tubing (U.S. Plastic Corp.). The solution flow rate was adjusted with a ball valve at the bottom of the reactor so that the pore space within the clay substrate was very open, indicating a fluidized condition. After flowing through the reactor 48, the feed solution flowed back into the mixing vessel 42. Just after leaving the reactor 48, operators used a valve 52 to measure dissolved oxygen (DO) content. The flow rate of oxygen into the mixing vessel was regulated so that DO concentration of the fluid was maintained at 8-16 ppm. Waste was extracted into a waste container 53.

The microcosm 40 was built to host and grow bacteria that would convert ammonium to nitrate at a very low pH. The initial bacterial source came from a local wastewater treatment plant (Rogers, Ariz.). A 450 ml sample taken from the aeration basin was added to the reactor 48. Then 350 ml of aeration basin supernatant solution (where solids had settled out) was also added. Then a 50 ml solution containing 0.115 g of ammonium chloride and 0.250 g of sodium bicarbonate was added and the reactor 48 was brought to volume with tap water. After a pump 51 was turned on, a feed solution 54 (20 L solution containing 2.137 g ammonium chloride, 8.76 g sodium bicarbonate and 0.040 g potassium phosphate) was added to the mixing vessel 42 at a rate of 7.6 ml/minute.

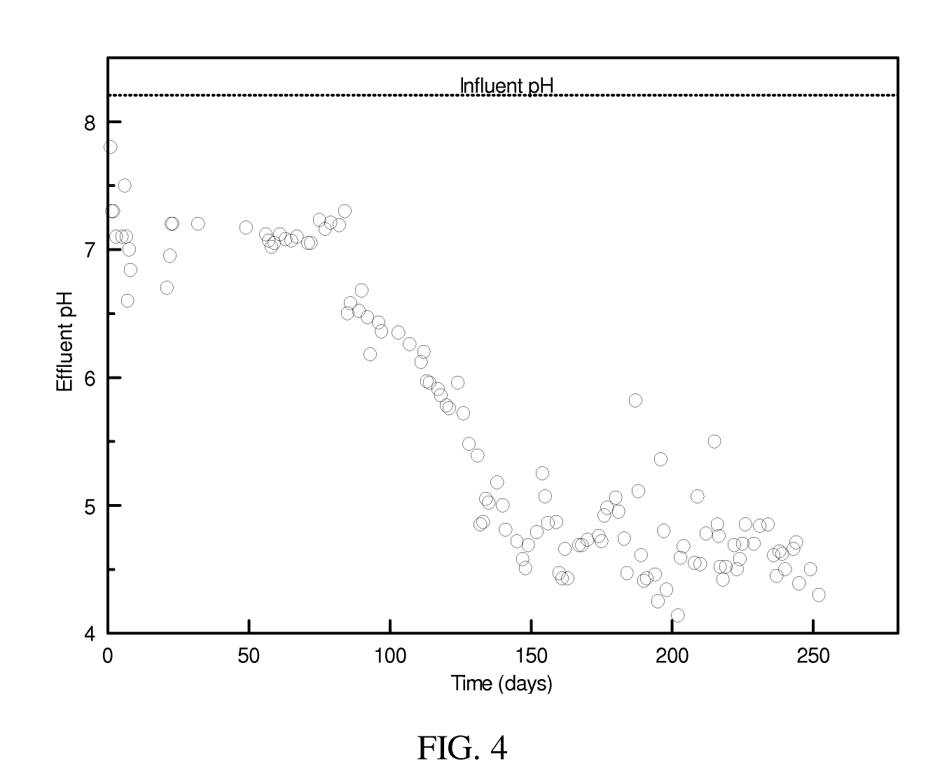

After a period of several weeks, when nitrate analysis of the waste effluent 53 indicated that greater than 95% of the ammonium was being converted to nitrate, the ammonium chloride concentration in the feed solution 54 was doubled. When nitrate analysis of the effluent showed that the majority of this solution was being nitrified, then the ammonium chloride concentration was increased again. After several months the 20 L feed solution 54 contained 17.1 g ammonium chloride, 80 g sodium bicarbonate and 0.080 g potassium phosphate. If one assumes all of this ammonium is nitrified and two moles of acidity are formed from each mole of ammonium, then the base/acid ratio with this amount of sodium bicarbonate and ammonium chloride is roughly 1.5, which causes the influent pH from the reactor 48 to be above 7 (as shown in FIG. 4). The pH of the feed solution (influent pH) was approximately 8.2. The temperature of the solution typically ranged from 30 to 32° C.

After the system 40 ran for several weeks at this high ammonium loading rate, the amount of sodium bicarbonate in the feed solution 54 was decreased with time in order to create acidic conditions. When the sodium bicarbonate was decreased from 80 g to 55 g, the pH of the effluent decreased from 7.1 to 6.5. This process was continued over a two month period until the amount of sodium bicarbonate added in the feed solution 54 was 50.1 g/20 L, which resulted in the molar ratio of base (moles sodium bicarbonate) to potential acidity (2 times moles ammonium chloride) to equal 0.94, at which point the effluent pH varied from 4.1 to 4.6.

Such low pHs in the effluent are very surprising since the influent pH was 8.2. These data indicate that nitrifying bacteria are generating large amounts of acidity. It should be noted that pH is a log scale, hence, a solution with a pH of 4.2 is 10,000 times more acidic than a solution with a pH of 8.2. All of this acidity was formed by nitrification. Nitrate analysis of the effluent indicated that 97% of the ammonium was being nitrified at this point, indicating that acid-tolerant nitrifying organisms were present and very active.

### EXAMPLE 2

### Field Study at Commercial Poultry Farm

A two-stage air scrubber was constructed and attached to one of the minimum ventilation fans at a commercial broiler farm in Madison County, Ariz. The two-stage scrubber had a configuration that was very similar (essentially the same) as the scrubber shown in FIGS. 1 and 2. The first stage or box of the scrubber removes the majority of dust, which is mainly manure, from the air. The second stage is for scrubbing ammonia. The dimensions of each box were 5′×5′×5′, although the size can be altered depending on air flow rates, which would determine the air residence time in the scrubber. Each scrubber has a reservoir which has a capacity of about 360 L. A simple float switch, similar to that in a toilet, controls the water level in both scrubbers. The scrubbers are equipped with heaters to keep the contents from freezing in the winter and to help achieve proper temperatures for bacterial growth.

Each scrubber can hold up to 8 rows of wooden slats at 45° angles. An inexpensive submersible pump is used to recirculate the water. Water from the pump is delivered into PVC pipes at the top of the scrubber which have a series of small holes (0.1 cm) in them. This system produces a water curtain. When air flow is high (i.e.—when the poultry house fans are on) the air turbulence causes the water to break up into fine droplets. Typically scrubbers on poultry houses that use nozzles to spray water or acid to scrub ammonia will clog when the scrubbing solution is being recycled. The scrubbers clog because of the large amount of particulate matter in the air that is picked up by the scrubber system. However, this design allows the scrubber to function without any clogging problems. The dust scrubber is also equipped with a removable screen that is above the reservoir to catch feathers.

A clay substrate of the type described in example 1 was used as inoculum for this experiment. Approximately 50 g of clay/biofilm was taken from the laboratory nitrifying reactor 48 and was applied to five slats in the ammonia scrubber. The substrate was applied in 1.5-2″ strips on the wooden slats which were covered in four layers of cheesecloth. Additional clay/biofilm was applied on a second row of slats; this time the microbial mixture was covered with nylon paint strainer so that a 2″ flap hung down below the slat to facilitate the growth of more biofilms. Another 50 g of the clay/biofilm mixture was added directly to the reservoir of the ammonia scrubber. When the ammonia scrubber reservoir was refilled with water a small amount of acid (300 ml of 1 N HCl) was added to reduce the water pH to around 7.0. A solution containing 17.1 g of ammonium chloride was also added to the reservoir so that the nitrifying bacteria had an ammonium source as soon as they were placed in the scrubber. This would supply roughly 12 mg NH₄—N/L.

The pumps in both scrubbers were set to run continuously. Likewise, the minimum ventilation fan on which the scrubber was attached was also set to run continuously. A Sigma automatic water sampler was used to take a 500 ml sample of the contents of the ammonia scrubber reservoir every 8 hours. Samples were analyzed for pH, electrical conductivity, ammonium, and nitrate. Temperature was also measured periodically.

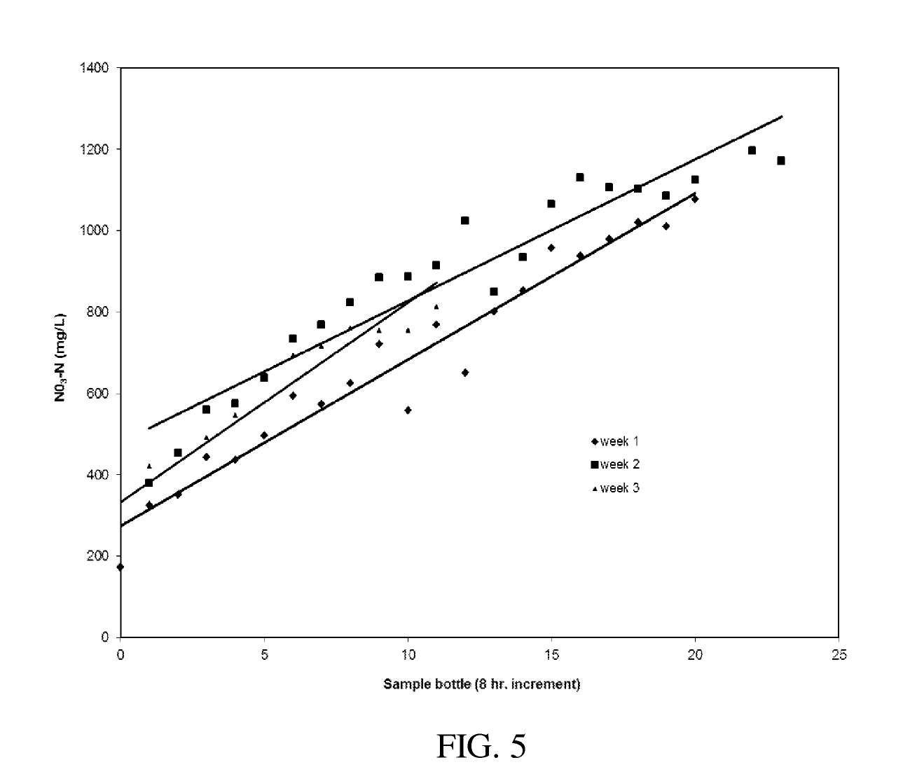

The scrubber ran for several days as described above, then the reservoir was drained to about 2.0″ (the depth of water when full was 6″), then refilled with water. Then the HCl and ammonium chloride was added and the pumps were turned back on. The ammonium chloride addition was probably not needed, since beginning concentration of ammonium in the ammonia scrubber was typically around 50 mg NH₄—N/L, indicating that the 2.0″ of remaining scrubber solution supplied the majority of the ammoniacal N at the beginning of each experiment. Likewise the small amount of acid added probably had little effect on pH. The nitrate-N concentration in the ammonia scrubber is shown in FIG. 5. Concentrations increased from around 200 mg N/L to over 1,000 mg N/L during the average study period. These data indicate that nitrification of ammonium was occurring.

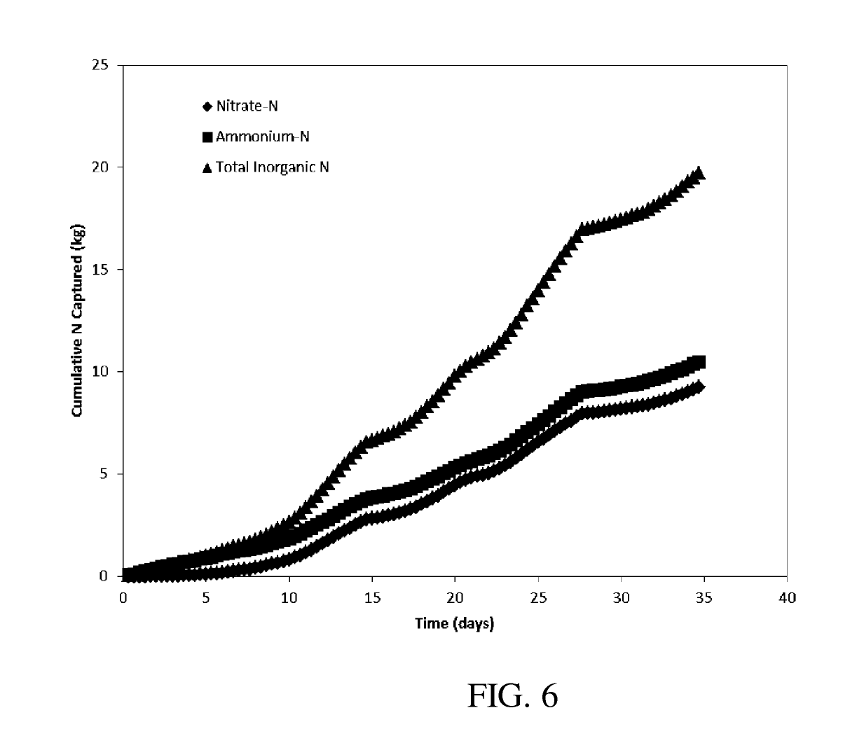

The mass of ammonium and nitrate captured over a five week period is shown in FIG. 6. Surprisingly, the scrubber captured a total of 19.8 kg of inorganic N (which is the sum of ammonium and nitrate). It should be noted that this trial was conducted in late July and August; a time when poultry growers turn on most, if not all, of their primary and tunnel fans in order to cool down the birds. As a result of this high ventilation rate, the ammonia concentration in the air in poultry houses during the hottest part of the summer is very low; typically around 2-5 ppm.

Research on the efficiency of this scrubber to capture ammonia that was conducted under controlled conditions in a shop with sulfuric acid in the ammonia scrubber showed there was a linear relationship between the concentration of ammonia in the air being scrubbed and the mass of N captured, with the rate of capture increasing from 25 g N/hr at 10 ppm NH₃ to 200 g N/hr at 75 ppm NH₃ (P. A. Moore, Jr., unpublished data). Although ammonia concentrations in poultry houses are variable, typically concentrations are around 25 ppm. Under such conditions the mass of N captured are much greater than that shown in FIG. 6. The amount of ammonium present in the scrubber was typically equal to or greater than the nitrate.

This was not the case with the laboratory microcosm study, where over 95% of the ammonium was converted to nitrate (Example 1). There are several possible explanations for this. One possible explanation relates to the differences in the two systems. In the laboratory microcosm, all of the solution was pumped through the clay (Filtralite) media, which held the bacteria. In the scrubber, only a fraction of the water would have contacted the strips of clay holding the bacteria.

Another big difference was the amount of bacteria present. The laboratory microcosm contained over one kg (wet basis) of clay substrate, whereas a total of only 100 g of clay substrate was added to the scrubber. However, the most important differences between the two systems as far as bacterial growth were concerned was the environmental conditions, such as oxygen content and temperature of the water, both of which were fairly constant and ideal under laboratory conditions. Dissolved oxygen was kept above 8 ppm and temperature was fairly constant at 30-32° C.

The scrubber system shown in FIGS. 1 and 2 is a static system that is more or less a batch reactor. If the animal containment facility were located on land used to grow row crops or forages, then a continuous flow scrubber system could be utilized. In this system water from a well, stream, river, lake, pond, reservoir, canal, ditch or from rain could be slowly metered into the dust and ammonia scrubbers. There are advantageous to growing the bacteria in the ammonia scrubber in a continuous flow system, primarily because the concentration of metabolites in the product solution stays relatively low. This product solution water could be then used to irrigate crops, which benefit from the higher nitrogen content.

For the foregoing reasons, it is clear that the method and apparatus described herein provides an innovative exhaust air scrubber system for animal containment facilities. The current system may be modified in multiple ways and applied in various technological applications. The disclosed method and apparatus may be modified and customized as required by a specific operation or application, and the individual components may be modified and defined, as required, to achieve the desired result.

Although the materials of construction are not described, they may include a variety of compositions consistent with the function described herein. Such variations are not to be regarded as a departure from the spirit and scope of this disclosure, and all such modifications as are obvious to one skilled in the art are intended to be included within the scope of the following claims.

What is claimed is:

1. An exhaust air scrubber system for an animal containment facility comprising an ammonia-removing scrubber, the ammonia scrubber being inoculated with acid-tolerant nitrifying bacteria.

2. The system of claim 1 wherein the acid-tolerant bacteria comprises an ammonia-oxidizing bacteria and a nitrate-oxidizing bacteria.

3. The system of claim 2 wherein the ammonia-oxidizing bacteria comprises an effective amount of Proteobacteria, Nitrosomonas, Nitrosococcus, or a combination thereof.

4. The system of claim 2 wherein the nitrate-oxidizing bacteria comprises an effective amount of Nitrobacter and/or Nitrospira or a combination thereof.

5. The system of claim 1 wherein the acid-tolerant bacteria are adapted to tolerate solutions having a pH value of about 4.1 to 7.0.

6. The system of claim 1 wherein the system is configured so that the acid-tolerant bacteria causes the removal of up to 95% of the volatile organic compounds (VOCs).

7. The system of claim 1 wherein the ammonia scrubber is inoculated with a sufficient quantity of acid-tolerant bacteria so that the ammonia scrubber yields a product solution having a pH value of about 8 or lower.

8. The system of claim 1 wherein the ammonia scrubber comprises a batch-type scrubber.

9. The system of claim 1 wherein the ammonia scrubber comprises a continuous-type scrubber.

10. The system of claim 1 wherein the ammonia scrubber is structured so that surfaces of the ammonia scrubber support substrates comprising the acid-tolerant bacteria, the ammonia scrubber being configured so that a scrubber solution interacts with the acid-tolerant bacteria to nitrify the ammonia and ammonium in the scrubber solution so that the scrubber solution and a product solution exhibits more acidity.

11. The system of claim 10 wherein the system is configured to cause an exhaust gas to be directed through the ammonia scrubber, the exhaust gas interacting with the scrubber solution to remove ammonia from the exhaust gas.

12. The system of claim 10 wherein the substrates comprise at least one slat.

13. The system of claim 12 wherein the ammonia scrubber is structured so that the acid-tolerant bacteria comprises a biofilm on the at least one slat.

14. The system of claim 13 wherein the ammonia scrubber is structured so that the biofilm grows on a clay medium and/or a medium comprising a support matrix.

15. The system of claim 14 wherein the support matrix compises a fibrous support matrix.

16. The system of claim 12 wherein the scrubber solution passes over and/or through the slat.

17. The system of claim 10 wherein the product solution and/or the scrubber solution is aerated.

18. The system of claim 10 wherein the acid-tolerant bacteria are cold tolerant, the product solution and the scrubber solution being maintained in a range of 5° C.-25° C.

19. The system of claim 10 wherein the product solution and/or the scrubber solution are heated.

20. The system of claim 1 the scrubber system further comprising a dust-removing scrubber.

21. The scrubber system of claim 20 wherein the dust-removing scrubber comprises a water-based scrubber solution.

22. The system of claim 20 wherein the dust-removing scrubber effectively reduces the alkalinity of the exhaust air.

23. The system of claim 20 wherein the dust removing scrubber effectively removes manure-based dust from the exhaust air.

24. The system of claim 20 wherein the system is structured so that the dust-removing scrubber is in series with the ammonia scrubber.

25. The system of claim 20 wherein an exhaust gas flows through the dust-removing scrubber and then to the ammonia scrubber.

26. The system of claim 20 wherein the ammonia scrubber and/or the dust scrubber is located inside the animal containment facility.

27. The system of claim 20 wherein the system is structured so that product solutions from both the dust-removing scrubber and the ammonia scrubber comprise effective fertilizers.

28. A method of removing ammonia from an animal containment facility's exhaust air, the method comprising the steps of:
(a) providing an ammonia scrubber inoculated with an effective amount of an acid-tolerant nitrifying bacteria;
(b) directing the exhaust air from the animal containment facility through the ammonia scrubber.

29. The method of claim 28 wherein, in step (a) the acid-tolerant bacteria comprises an ammonia-oxidizing bacteria, and a nitrate-oxidizing bacteria.

30. The method of claim 29 wherein the ammonia-oxidizing bacteria comprises an effective amount of Proteobacteria, Nitrosomonas, Nitrosococcus, or a combination thereof.

31. The method of claim 29 wherein the nitrate-oxidizing bacteria comprises an effective amount of Nitrobacter and/or Nitrospira or a combination thereof.

32. The method of claim 28 wherein the acid-tolerant bacteria are adapted to tolerate fluids having a pH value of about 4.1 or higher.

33. A method of making an ammonia scrubber, the method comprising the steps of:
(a) providing a housing;
(b) positioning at least one substrate within the housing; and,
(c) inoculating the at least one substrate with an effective amount of an acid-tolerant nitrifying bacteria.

34. The method of claim 33 wherein the substrate supports a biofilm comprising the acid-tolerant bacteria.

35. The method of claim 33 further comprising:
(d) circulating a scrubber solution so that the acid-tolerant bacteria acidifies the scrubber solution.

36. The method of claim 33 wherein, in step (c) the acid-tolerant bacteria comprises an ammonia-oxidizing bacteria, and a nitrate-oxidizing bacteria.

37. The method of claim 36 wherein the ammonia-oxidizing bacteria comprises an effective amount of Proteobacteria, Nitrosomonas, Nitrosococcus, or a combination thereof.

38. The method of claim 36 wherein the nitrate-oxidizing bacteria comprises an effective amount of Nitrobacter and/or Nitrospira or a combination thereof.

39. The method of claim 33 wherein the acid-tolerant nitrifying bacteria are also cold tolerant.

40. The method of claim 33 wherein the scrubber is located inside an animal rearing facility.
